(19) United States
(12) Patent Application Publication
(10) Pub. No.: US 2012/0130449 A1
(43) Pub. Date: May 24, 2012

(54) COCHLEAR IMPLANT APPARATUS AND METHODS

(76) Inventors: Robert Carlyon, Cambridgeshire (GB); Oliver Macherey, Marseille (FR); Jan Wouters, Holsbeek (BE); Astrid Van Wieringen, Heverlee (BE)

(21) Appl. No.: 13/379,290

(22) PCT Filed: Jun. 21, 2010
(86) PCT No.: PCT/GB2010/051020

§ 371 Date: Feb. 13, 2012

(30) Foreign Application Priority Data
Jun. 23, 2009 (GB) 0910811.9

(51) Int. Cl. A61F 11/04

(52) U.S. Cl. 607/57

(57) ABSTRACT

The invention relates to cochlear implants and to improved methods of operating such implants. We describe cochlear implant apparatus comprising an audio signal processing unit with a wired or wireless coupling to an implantable cochlear stimulation device, wherein said audio signal processing unit has an audio input to receive an audio signal representing a sound, wherein said implantable cochlear stimulation device includes an electrical pulse generator coupled to an electrode array for intracochlear stimulation, wherein the apparatus is configured to apply stimulation to said electrode array to represent said sound, wherein said stimulation comprises application of an electrical pulse across two intracochlear electrodes of said electrode array, and wherein said electrical pulse has a pulse waveshape which is asymmetric under an operation comprising inverting the waveshape about a zero level and then time reversing the waveshape.

### FIELD OF THE INVENTION

This invention relates to cochlear implants and to improved methods of operating such implants.

### BACKGROUND TO THE INVENTION

Broadly speaking a cochlear implant comprises two main parts, an external speech processor and an internal, implanted cochlear stimulation device which receives signals and power from the external speech processor and which drives an electrode array. It is most useful in people having severe to profound sensorineural hearing impairment and operates by stimulating the auditory nerve fibres electrically. The cochlea itself is a conical helical chamber of bone including the fluid-filled scala tympani and scala vestibuli separated by the basilar membrane on which sound-induced mechanical waves travel. The base of the cochlea (nearest the round and oval windows) is relatively stiff and responsive to high frequencies; the apex of the cochlea is tuned to receive lower frequencies (tonotopic organisation); when functioning, hair cells respond to the motion of fluid to generate electrical signals. Generally the electrode array is implanted into the scala tympani.

Referring to FIG. 1A, this shows an outline block diagram of cochlear implant apparatus 100 comprising a microphone 102 coupled to an audio signal processing unit 104, which in turn is coupled to a transmitter 106; these items are carried externally by a user. The transmitter transmits a signal through the skin of the user to a receiver 108 which is coupled to a cochlear stimulation device 110 which drives an intracochlear electrode 112 with respect to an extracochlear reference electrode 114 or to another intracochlear electrode. The receiver 108, stimulation device 110, and electrodes 112, 114 are implanted inside a user's head; in some systems a wired percutaneous connection may be employed instead of the wireless transmitter-receiver. Devices of this general type are available from, inter alia, Cochlear Limited, Advanced Bionics Corp, and Med-El GmbH; each of these three companies has filed a number of patent applications to which reference may be made for background prior art (see, for example, Advanced Bionics patents U.S. Pat. No. 6,219,580; U.S. Pat. No. 7,149,583, and U.S. Pat. No. 7,515,966).

It is known to employ a number of different strategies when processing speech in the audio signal processing unit 104, but broadly speaking these generally employ a set of band pass filters which divide the captured speech into a number of different frequency bands, the signal levels in these bands then being used to control stimulation of the intracochlear electrodes. FIG. 1B shows an example decomposition of a speech waveform in this manner. A variant on this approach is to identify the fundamental and one or more formant frequencies in the captured speech (in effect modelling the vocal tract), using these to control the stimulation of the electrodes. The number of intracochlear electrodes in present devices typically varies between six and twenty two; the number of band pass filters may be more than this. Generally some compression is applied to the envelope of the band pass filtered signal because of the limited dynamic range of the electrical stimulation. Generally pulsed electrical stimulation is applied to the electrodes (although some systems make provision for stimulation using an arbitrary quasi-analogue wave form). Since stimulation of multiple electrodes simultaneously can cause unwanted side effects such as electrical or neural interactions, it can be preferable to simulate the electrodes sequentially but repetitively (continuous interleaved sampling). However the degree of stimulation appears to be related to the amount of charge delivered and therefore a strategy which requires short pulses may also require relatively high peak currents deliverable by a current driver in the cochlear stimulation device.

In general, and as illustrated in FIG. 1A, so-called monopolar stimulation is employed—that is an approach in which an intracochlear electrode 112 is stimulated with respect to an extracochlear reference or return electrode 114. That is, a pulse of current from a constant current generator in the cochlear stimulation device 110 is applied to one of the intracochlear electrodes of the electrode array 112 and this current flows through the user's tissue back to reference or return electrode 114. However, it is also known to apply bipolar stimulation to the intracochlear electrode array 112: in bipolar stimulation a pulse of current is applied so that current flows between two of the intracochlear electrodes—that is the reference or return electrode is inside the cochlea. With bipolar stimulation the pair of stimulated electrodes may be adjacent one another in the electrode array or separated by one or more intermediate electrodes (but nonetheless still relatively close to one another)—these different types of bipolar stimulation may be referred to a BP, BP+1, BP+2 and so forth where BP+n refers to a number n of intermediate electrodes between the two electrodes across which the current drive is applied. It is further known to apply tripolar stimulation which, for the purposes of this specification, is considered as a subset of bipolar stimulation since with tripolar stimulation the pulse of current is applied across three intracochlear electrodes of the electrode array, a central electrode and two adjacent or nearby reference or return electrodes each receiving approximately half the return current.

Generally speaking, bipolar stimulation is considered less efficient than monopolar stimulation because it appears less effective, probably because there is a relatively direct current path between two closely spaced intracochlear electrodes of the electrode array so that a higher drive current and/or longer than preferable pulse duration is needed for the same level of auditory nerve stimulation.

Separately to whether monopolar or bipolar stimulation is employed, monophasic or biphasic current pulses may be employed to drive an introcochlear electrode of the electrode array. It is important to carefully distinguish between these different terms of art—monopolar/bipolar refers to the electrodes across which the current drive is applied, as described above; monophasic/biphasic refers to the shape of the current pulses applied, as described further below.

FIG. 2, which is taken from the inventor's paper, “Asymmetric pulses in cochlear implants: effective pulse shape, polarity, and rate”, O. Macherey, A Van Wieringen, R P Carlyon, J M Deeks, J Wouters, JARO 7:253-266 (2006) shows examples of different shapes of current pulse which may be employed. FIG. 2A shows a symmetric biphasic current pulse in which the positive phase of the pulse, referred to as the anodic phase, comes first. FIG. 2B shows alternating monophasic pulses; FIG. 2C shows pseudomonophasic pulses in which the positive (anodic) phase of the pulse comes first; FIG. 2D shows pseudomonophasic cathodic (negative) first pulses.

For safety reasons there is a requirement that the overall charge delivered by a pulse of current is substantially zero (so that there is no build up of charge which could cause nerve damage). It can be seen that this is satisfied by the symmetric biphasic pulses of FIG. 2A. Generally cochlear implant devices use symmetric biphasic current pulses. In our pseudomonophasic pulses again the overall charge delivered is substantially zero but one phase of the pulse is of shorter duration and higher amplitude than the other so that the pulse has some of the characteristics of a monophasic pulse. Thus where the short, high-amplitude portion of the pulse is positive we refer to this as an anodic pseudomonophasic pulse; where it is negative we refer to a cathodic pseudomonophasic pulse. Optionally there may be a delay between the short, high amplitude phase of the pulse and the longer, lower amplitude phase of the pulse, as shown in FIGS. 2E and 2F; we refer to these as delayed pseudomonophasic pulses. FIGS. 2G and 2H show alternating pseudomonophasic pulses (FIG. 2H, alternating delayed pseudomonophasic pulses), as illustrated an anodic pulse followed by a cathodic pulse. Pseudomonophasic pulses also include the time-reversed versions of the waveshapes shown in FIGS. 2c-2f. In the illustrated example the ratio of short:long pulse phases is 1:8, but this can be varied. For example, under the constraint of zero total delivered charge the ratio may be varied continuously between, say, 10:1 to 1:1 to 1:10 to vary between, say, an anodic pseudomonophasic pulse, a symmetric biphasic pulse, and a cathodic pseudomonophasic pulse. The pulse durations and current amplitudes can vary substantially, but an example pulse duration is between 10 μs and 100 μs (for the short, high amplitude portion of a pseudomonophasic pulse): the amplitude can vary between, for example, 100 μA and 1000 μA although a cochlear stimulation device may have the ability to drive currents up to a few milliamps.

Some cochlear stimulation devices can only produce symmetric biphasic pulses, albeit of variable amplitude, timing, and phase (positive first or negative first). Other devices enable the audio signal processing unit to control the shape of a current pulse wave form applied; there may even be provision for applying an analogue current wave form. Similarly, a device may have provision for both monopolar and bipolar stimulation, selected under control of the audio signal processing unit. Some devices have just a single current driver; others have multiple current drivers, for example 1 per electrode, to enable simultaneous stimulation of two or more electrodes with different currents.

Advanced Bionics has, on its website, a description of “active current steering” in which current is delivered simultaneously to adjacent intracochlear electrodes using monopolar stimulation with one current source per electrode. Control of the proportion of the current delivered, in phase, to each electrode of the pair enables the locus of stimulation to be steered between the two electrodes. Some speech processing strategies making use of this steering ability to address “virtual electrodes” positioned between real, physical electrodes are described in U.S. Pat. No. 7,515,966.

Many different types of electrode array are known. Generally a longitudinal carrier, bearing a number of platinum or platinum/iridium electrodes, is provided, each electrode having a separate lead. However, ball electrode arrays are also known in which multiple ball electrodes, optionally embedded in a carrier, are used in an array. The extracochlear electrode is typically a plate, but can also be another type of electrode or two electrodes in parallel. As previously mentioned, typically the electrode array is inserted into the scala tympani so that it is able to stimulate nerve fibres towards both the base, middle, and apex of the cochlea.

A cochlear implant generates two different types of pitch percept in a user, a temporal pitch percept and a place pitch percept. These two cues to pitch are perceptually independent and correspond to two separate perceptual dimensions. The rate of stimulation of an electrode generates the temporal pitch percept, and the location of the electrode along the cochlea generates the place pitch percept. In broad terms, the temporal pitch percept relates to the fundamental frequency of the sound and the place pitch percept to the spectral envelope; it is possible to have a high place pitch percept in combination with a low temporal pitch percept, and vice-versa. Normal hearing uses both of these pitch percepts, although the temporal pitch percept is most important up to about 2 kHz and is apparently also responsible for the phenomenon of the “missing fundamental”.

Although increasing the repetition rate of a stimulus applied to an electrode increases the temporal pitch heard, this increase is usually subject to an upper limit of around 300-400 Hz. That is, experiments using simple pulse strains show that pitch increases with pulse rate only up to 300-400 Hz, which is poor but adequate for speech since the information content of speech is mostly in the lower frequencies. There is, however, a desire to improve this upper limit and, more generally, to improve both temporal and place pitch percepts.

Recent phsysiological data (Middlebrooks & Snyder 2009—Middlebrooks, J. and Snyder, R. (2009), “Enhanced Transmission of Temporal Fine Structure Using Penetrating Auditory Nerve Electrodes,” Association for Research in Otolaryngology, 32nd Midwinter Research Meeting Baltimore, Md., USA) showed that neurons in the guinea pig that were driven by stimulation at the apex were able to follow higher stimulation rates (up to 600 pps, pulses per second) than those driven by more basal stimulation, which showed degraded phase-locking at 300 pps. Nevertheless, previous studies comparing temporal pitch discrimination at different cochlear locations by human listeners did not find better performance with apical stimulation. In Macherey et al 2006 (“higher sensitivity of human auditory nerve fibres to positive electrical currents”, O Macherey, R P Carlyon, A Van Wieringen, J M Deeks, J Wouters, J A R09:241-251 (2008)) the inventors found that, with monopolar stimulation, less current was required to reach mcl (most comfortable levels) in ci (cochlear implant) users with an anodic pseudomonophasic pulse than with a cathodic pseudomonophasic pulse. As previously mentioned, whilst each of these pulses contains two opposite polarity phases, we refer to “anodic” as the one having a short, high amplitude anoidic phase and to “cathodic” as the one with a short, high-amplitude cathodic phase. This result was surprising as animal experiments have shown that cathodic current flows are more effective than anodic ones in eliciting neural responses; the inventors showed that the human auditory system exhibits the opposite pattern, and that at an equal stimulus level the anodic phase yielded the larger response.

Thus, to recap, most contemporary cochlear implants stimulate the cochlea with symmetric biphasic pulses consisting of a first phase of one polarity followed by a second phase of opposite polarity. In addition, speech processing strategies currently implemented in those devices use monopolar stimulation where each intracochlear electrode (so-called “active” electrodes) is stimulated with reference to an extracochlear electrode (so-called “return” electrode). We will describe techniques for improving pitch perception by cochlear implant users.

### SUMMARY OF THE INVENTION

According to a first aspect of the invention there is therefore provided cochlear implant apparatus comprising an audio signal processing unit with a wired or wireless coupling to an implantable cochlear stimulation device, wherein said audio signal processing unit has an audio input to receive an audio signal representing a sound, wherein said implantable cochlear stimulation device includes an electrical pulse generator coupled to an electrode array for intracochlear stimulation, wherein the apparatus is configured to apply stimulation to said electrode array to represent said sound, wherein said stimulation comprises application of an electrical pulse across two intracochlear electrodes of said electrode array, and wherein said electrical pulse has a pulse waveshape which is asymmetric under an operation comprising inverting the waveshape about a zero level and then time reversing the waveshape.

In embodiments an asymmetric pulse waveshape applied with bipolar stimulation provides a number of advantages, described more fully below. The arrangement enables stimulation of neurons closer to the apex of the cochlea, enabling stimulation of a lower place pitch percept than would otherwise be possible, which is particularly useful because often the electrode array is not fully inserted within the cochlea. The arrangement also facilitates attaining a higher upper limit of temporal pitch, for example the perceived temporal pitch increasing up to a stimulation rate of about 700-800 pulses per second. The arrangement also enables selective stimulation of neurons, which the inventors term “neural steering”, by exploiting the polarity dependence of the neural response.

The asymmetry of the waveshape of the pulse may alternatively be expressed as the pulse being asymmetric under a 180° rotation about the mid-point of the waveform. In embodiments the waveform may be pseudo monophasic, triphasic, or quadriphasic. In embodiments the pulse waveshape is configured such that when integrated (from 0) the integrated value of the waveshape has a greater excursion away from the zero level in one direction, more particularly the positive or anodic direction, than in the other direction (albeit the total, integrated value of the waveform is substantially zero so that substantially zero total charge is delivered). This may be achieved using a pseudomonophasic waveshape with a first, short duration high amplitude portion followed by a second lower amplitude longer duration portion (here “first” does not refer to first-in-time ie. the initial phase of the waveshape may comprise either the first or the second portions of the waveshape). Alternatively a triphasic waveform may be employed in which the short, high amplitude portion is between two longer lower amplitude portions. Still further alternatively a triphasic waveform may be used with three phases of the substantially same amplitude, but with different durations (shorter for the first and third phases than for the second). Some cochlear implants only support the use of symmetric biphasic pulses, in which case it is possible to synthesise a suitable asymmetric waveshape by controlling such a device to produce a pair of symmetric biphasic pulses, one inverted with respect to the other.

The asymmetric waveshape is applied using bipolar (which includes tripolar) stimulation—that is the current pulse is applied across a pair of intracochlear electrodes rather than between one (or more) intracochlear electrodes and an extracochlear reference or return electrodes. Only partial bipolar stimulation may be employed, in which a proportion of the current is returned via an extracochlear electrode. The intracochlear electrodes across which the pulse is applied are preferably close to one another, for example separated by 0, 1, 2 or 3 intermediate electrodes of the electrode array or by less than 3.5 mm, 2.5 mm or 1.5 mm.

In embodiments the electrical pulse comprises a pulse of current and the waveshape defines the current drive applied rather than the applied voltage. In embodiments the audio signal processing unit controls the electrical pulse generator in the implantable cochlear stimulation device to define the (asymmetric) waveshape of the current pulse; additionally or alternatively however the pulse generator may itself be configured to generate an appropriate asymmetric current drive waveshape.

As previously mentioned, the asymmetric waveshape is configured such that integration of the waveshape results in a greater excursion in one direction (the positive or anodic direction) away from the zero level than in the other direction. In embodiments the apparatus is configured such that the portion of the asymmetric waveshape above the zero level, that is the positive or anodic portion of the waveshape, is applied to one of the intracochlear electrodes of the array which is (when the electrode array is implanted) closest to the apex of the cochlea. This enables nerve fibres closer to the apex to receive more stimulation than those further away from the apex of the cochlea, in broad terms facilitating selective stimulation of neurons closer to the apex of the cochlea. The inventors have found, experimentally, that this is able to produce a lower place pitch percept than would otherwise be the case. Further, and surprisingly, the neurons which are responsive to lower place pitches appear able to respond more rapidly to stimulation and therefore pulse stimulation applied to these neurons is able to produce increasing temporal pitch percepts with increased pulse rate up to a higher limit than previously, for example up to a pulse rate of above 600 Hz, 700 Hz or in some subjects, 800 Hz.

Thus asymmetrical waveshapes of the type described above are particularly useful for stimulating an electrode of the electrode array which is at the apex end of the array or within 4, 3, 2, 1 electrodes of the apex. Other waveshapes may be better for stimulating electrodes near the base of the cochlea. Thus in embodiments the asymmetric pulse waveshape is used selectively to stimulate one or more electrodes at the most apical end of the electrode array. Selective use of the asymmetric waveshape and/or bipolar stimulation near the apical end of the electrode array is also practically useful because it can be difficult to provide loud sounds with bipolar stimulation because there is a limit on the maximum available current drive, and although this can be addressed by providing a lower current for longer, this reduces the overall maximum available pulse rate, in particular in devices which apply an interleaved rather than simultaneous pulse drive strategy.

The above described asymmetric current drive pulse waveshapes, together with the observation that it is the positive/anodic portion of the waveshape which is apparently most effective at stimulating a neuron, enable what the inventors term “neural steering” of the place pitch percept. As previously described, the waveshape is applied across a pair of intracochlear electrodes of the electrode array, one of these acting as the drive electrode, the other as the reference for return electrode. The asymmetric waveshape has one polarity (positive anodic) which is larger than the other, more specifically which has an integrated value with a greater excursion from the zero level than the other. With bipolar (or tripolar) stimulation one of the intracochlear electrodes is driven with reference to one (or more) of the other intracochlear electrodes. With a “normal” asymmetric waveshape as previously described, the neural stimulation is greatest near the driven electrode; if this waveshape is inverted then the neural stimulation is greatest near the reference or return intracochlear electrode (in effect, the driven electrode is driven negative with respect to the reference/return electrode or, alternatively, the reference/return electrode is driven positively with respect to the driven electrode. The inventors have recognised that by varying the waveshape between its normal and inverted versions the effectiveness of the neural stimulation can be varied in location between a position close to the driven electrode and a position close to the reference/return electrode (by moving the location of anodic stimulation), thereby moving the place pitch percept. It will be appreciated that this is performed by changing the effective anodic stimulation location (integrated positive value) of the asymmetric waveshape rather than simply by applying a current to one or other of two intracochlear electrodes with respect to an external extracochlear electrode.

The above techniques also facilitate alternative signal encoding techniques in the audio signal processing unit, to take advantage of one or more of the increased accuracy of place pitch percept generation, the reduced lower limit of place pitch percept, and the increased upper limit of temporal pitch percept. For example the fundamental frequency of a speech signal determined by the audio signal processing unit may be encoded using a technique as described above when this fundamental frequency is greater than a threshold frequency, for example 300, 400, 500, 600 or 700 Hz, more particularly using most apical electrode stimulation for high fundamental frequencies and in embodiments an alternative form of stimulation for lower fundamental frequencies. In general the audio signal processing unit may be configured to determine at least one parameter representing a percept of the sound, and then bipolar stimulation with an asymmetric pulse waveshape as described above may be used to encode a first set of values of this parameter and a second, different pulse waveshape may be used to encode a second set of values of this parameter. This parameter may define, for example, a temporal pitch percept, for example fundamental frequency, in which case either high frequencies or all frequencies are encoded using asymmetric wave form bipolar stimulation; and/or the parameter may represent a low place frequency percept and/or be dependent on a level of harmonics or formants in a speech signal. Although in general the audio signal processing unit will operate on a signal from a local microphone, additionally or alternatively a line input may also be provided.

Thus in a related aspect the invention provides an audio signal processing unit for a cochlear implant, the signal processing unit comprising: an input to receive an audio signal representing a sound; an output to provide a control signal to an implanted cochlear stimulation device; data memory to store audio data for processing; program memory storing processor control code; and a digital signal processor, coupled to said program memory to load and implement said code, coupled to said data memory, and coupled between said input and said output to provide said control signal responsive to said audio input to create a perception of said sound in a user; and wherein said code comprises code for controlling said digital signal processor to generate audio signal representation data defining a representation of said audio signal as a set of pulses for bipolar driving of electrodes of said implanted cochlear stimulation device, wherein said representation data comprises, for a said pulse, a combination of data defining a pulse waveshape and data defining a pair of intracochlear electrodes to which said pulse waveshape is to be applied, wherein said data defining said pulse waveshape comprises data defining a waveshape to said electrode array to represent said sound which is asymmetric under an operation comprising inverting the waveshape about a zero level and then time reversing the waveshape, and to code control said control signal responsive to said audio signal representation data.

Such a unit may be employed in conjunction with an existing, implanted cochlear stimulation device since many such devices are sufficiently flexible to be controllable to employ the techniques described herein. In embodiments the unit may be configured to employ bipolar (or partial bipolar) stimulation only at the apex and to use monopolar stimulation for all the other electrodes. This makes the strategy easier to implement because one can use a long phase for the bipolar channel and short phases for the other monopolar channels, thereby allowing nonsimultaneous activation of the electrodes, facilitating increased stimulation rates. In embodiments the audio signal processing unit may process an audio input to generate a separate channel of data representing a fundamental frequency of the input. This data channel may then be employed to control a temporal pitch (pulse rate) channel of system whilst the original signal or the remainder of the signal is used to determine stimulation for a place pitch percept.

Thus the invention also provides an audio signal processing unit for a cochlear implant, the signal processing unit comprising: an input to receive audio signal data representing a sound; an output to provide a control signal to an implantable cochlear stimulation device; data memory to store audio signal data for processing; program memory storing processor control code; and a digital signal processor, coupled to said program memory to load and implement said code, coupled to said data memory, and coupled between said input and said output to provide said control signal responsive to said audio input to create a perception of said sound in a user; and wherein said code comprises code for controlling said digital signal processor to input or generate audio signal data defining a representation of said sound comprising two channels of data, a first channel representing a fundamental frequency of the input and a second channel representing the original signal or the remainder of the signal; and to send said two channels of data to said cochlear stimulation device to control said device to generate a temporal pitch percept from said first channel of data and to generate a place pitch percept or to convey other information from said second channel of data.

The invention also provides a method of processing data for a cochlear implant, and corresponding signal processor control code stored on a carrier such as a disk or memory, the method comprising generating audio signal data defining a representation of said sound comprising two channels of data, a first channel representing a fundamental frequency of the input and a second channel representing the original signal or the remainder of the signal; and sending said two channels of data to a cochlear stimulation device to control the cochlear stimulation device to generate a temporal pitch percept from said first channel of data and to generate a place pitch percept or to convey other information from said second channel of data.

In embodiments a separate processing unit, couplable to said audio signal processing unit, is used to generate the two channels of data. For example the audio signal data, which may be music, may be processed in advance, off-line, by for example a computer program, and the output of this processing may provide two channels of information to the implant, one with F0 and one with “regular” information. Thus one aspect of such embodiments of the invention is an implant processing unit configured so that it is able to process these two channels appropriately, rather than there necessarily being a separate processing unit to extract these two channels in the first place (although in other embodiments the audio signal processing unit may generate these two data channels). Thus, for example, a CI (cochlear implant) user may simply buy a CD or download a .wav file or .mp3 that has been processed in this way—or alternatively may run an existing sound file through a computer program to produce another sound file—and which they would input to the implant.

The invention also provides an audio signal processing unit as described above in combination with an implantable cochlear stimulation device to stimulate an electrode array to apply bipolar stimulation comprising a current drive waveshape as described above across two intracochlear electrodes.

However, also as previously mentioned, the implantable cochlear stimulation device may itself be configured to apply bipolar stimulation using an asymmetric current drive waveform as described above.

Thus in a further, related aspect the invention provides an implantable cochlear stimulation device, the device comprising: a signal input to receive a control signal from an external audio signal processing unit; at least one pulse generator to generate an electrical pulse; an electrode array for intracochlear stimulation coupled to said at least one pulse generator; and a controller coupled to said signal input and to said at least one pulse generator; and wherein said device is configured to apply stimulation comprising application of said electrical pulse across two intracochlear electrodes of said electrode array, wherein said electrical pulse has a pulse waveshape which is asymmetric under an operation comprising inverting the waveshape about a zero level and then time reversing the waveshape.

The invention further provides a method of stimulation of a cochlear implant, the method comprising stimulating a first intracochlear electrode with reference to a second intracochlear electrode using an electrical pulse such that a current flows from said first to said second intracochlear electrode via the cochlea, and wherein said stimulating uses an electrical pulse having waveshape which is asymmetric under an operation comprising inverting the waveshape about a zero level and then time reversing the waveshape.

The invention still further provides a method of controlling a cochlear implant, the cochlear input having: an input to receive a control signal from a remote device; at least one electrical pulse generator, coupled to said input to generate an electrical pulse responsive to said control signal; and an electrode array, said electrode array comprising a generally longitudinal support bearing electrodes at intervals along said support, to apply said electrical pulse to the cochlea of a user via one or more of said electrodes; the method comprising: applying a bipolar said electrical pulse to said cochlea using said electrode array to cause a perception of sound wherein, said applying of said bipolar pulse comprises applying a current drive to a first of said electrodes of said electrode array and providing a return path for said current drive via a second of said electrodes of said electrode array; and wherein the method further comprises: using a waveshape for said bipolar electrical pulse which is asymmetric when inverted about a zero drive level and then time reversed.

The invention also provides corresponding apparatus comprising means configured to implement such a method, for example a signal processor in combination with stored processor control code in non-volatile memory.

Thus the invention further provides processor control code to implement the above-described methods, for example on a digital signal processor (DSP). The code may be provided on a carrier such as a disk, CD- or DVD-ROM, or in programmed memory such as read-only memory or non-volatile memory such as Flash memory (Firmware). Code (and/or data) to implement embodiments of the invention may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog (Trade Mark) or VHDL (Very high speed integrated circuit Hardware Description Language). As the skilled person will appreciate such code and/or data may be distributed between a plurality of coupled components in communication with one another.

The invention still further provides cochlear implant apparatus including an audio signal processing unit having an input to receive an audio signal representing a sound, an output to provide a control signal to an implanted cochlear stimulation device, data memory to store audio data for processing, programme memory storing processor control code, and a digital signal processor, coupled to the programme memory to lower an implement to the code, coupled to the data memory, and coupled between the input and the output to provide the control signal in response to the audio input to create a perception of the sound in a user, and wherein the programme memory comprises non-volatile memory storing the above mentioned processor control code.

In another aspect the invention provides cochlear implant apparatus configured to adjust a location of neural stimulation, the apparatus including an audio signal processing unit, the audio signal processing unit comprising: an input to receive an audio signal representing a sound; an output to provide a control signal to an implanted cochlear stimulation device having an intracochlear electrode array; data memory to store audio data for processing; program memory storing processor control code to control operation of the audio signal processing unit; and a digital signal processor, coupled to said program memory to load and implement said code, coupled to said data memory, and coupled between said input and said output to provide said control signal in response to said audio input to create a perception of said sound in a user; wherein said cochlear implant apparatus is configured to drive first and second intracochlear electrodes of said intracochlear electrode array with a current drive, wherein said current drive has an adjustable waveshape to adjust said location of neural stimulation.

Preferably the intracochlear electrodes are driven such that a substantially equal and opposite currents flow through said electrodes. In embodiments the cochlear implant apparatus is configured by the processor control code stored in the (non-volatile) program memory. In embodiments the current drive waveshape is adjustable between a first version of the waveshape and a second inverted version of the waveshape. Thus in embodiments the current drive waveshape has first and second portions respectively above and below a zero level of the waveshape, and the current drive waveshape is adjustable to adjust an amplitude and duration of one of said first and second portions with respect to the other subject to a constraint that a total integrated value of the waveshape above said zero level is substantially equal to a total integrated value of the waveshape below said zero level.

The invention also provides a method of adjusting the location of neural stimulation by cochlear implant apparatus, the method comprising driving first and second intracochlear electrodes of said intracochlear electrode array with a current drive pulse, and adjusting a shape of said current drive pulse to adjust said location of neural stimulation.

The invention also provides cochlear implant apparatus comprising means, in particular a processor with associated control code, to implement the above described method. In embodiments the first and second intracochlear electrodes of said intracochlear electrode array are driven such that a substantially equal and opposite currents flow through the electrodes. Optionally, however, a proportion of the current may be returned via an extracochlear electrode.

The invention also contemplates substituting, for the definition of an asymmetric waveshape in the above statements of invention, a definition in which the waveshape is configured such that integration of the waveshape results in a greater excursion in one direction away from the zero level (the positive or anodic direction) than in the other direction (the negative or cathodic direction).

### BRIEF DESCRIPTION OF THE DRAWINGS

These and other aspects of the invention will now be further described, by way of example only, with reference to the accompanying figures in which:

FIGS. 1A and 1B show, respectively, a block diagram of typical cochlear implant apparatus, and an illustration of coding of a synthetic vowel (pitch=100 Hz) by an implant showing that only slow fluctuations are encoded;

FIG. 2 shows cochlear implant pulse shapes;

FIGS. 3A and 3B show, respectively, a technique for focusing stimulation in cochlear implants, and measured “upper limits” of pitch using different cochlear implant stimulation techniques;

FIG. 4 shows cochlear implant waveshapes;

FIG. 5 shows a graph illustrating the percentage of times a bipolar pseudomonophasic anodic stimulation relative to the most apical electrode of the implant was judged lower in pitch than a symmetric biphasic pulse applied to the same bipolar channel, for three different pulse rates;

FIGS. 6A and 6B illustrate results of a pitch ranking experiment for two subjects S1 (FIG. 6A) and S2 (FIG. 6B);

FIGS. 7A and 7B show neural steering with two different pulse shapes, respectively pseudomonophasic and quadriphasic;

FIG. 8 shows a block diagram of cochlear implant apparatus including stored processor control code to implement an embodiment of the invention;

FIG. 9 illustrates results of a pitch ranking experiment for four subjects S2, S3, S4, S7, for a range of different stimuli similar to those shown in FIG. 7A; and

FIG. 10 shows further measured “upper limits” of pitch using different cochlear implant stimulation techniques.

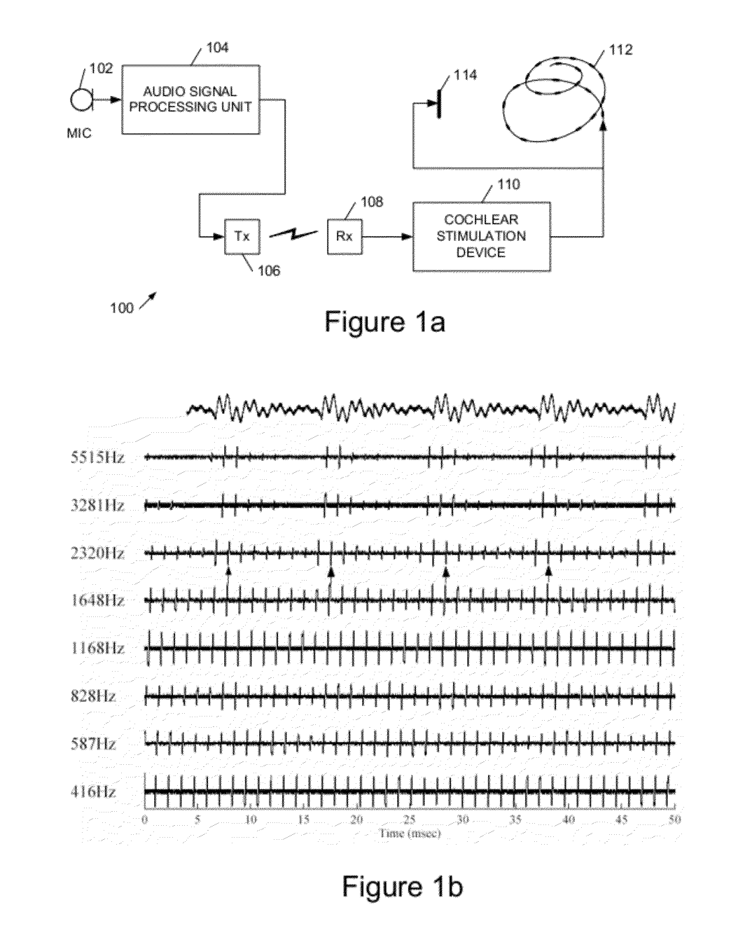

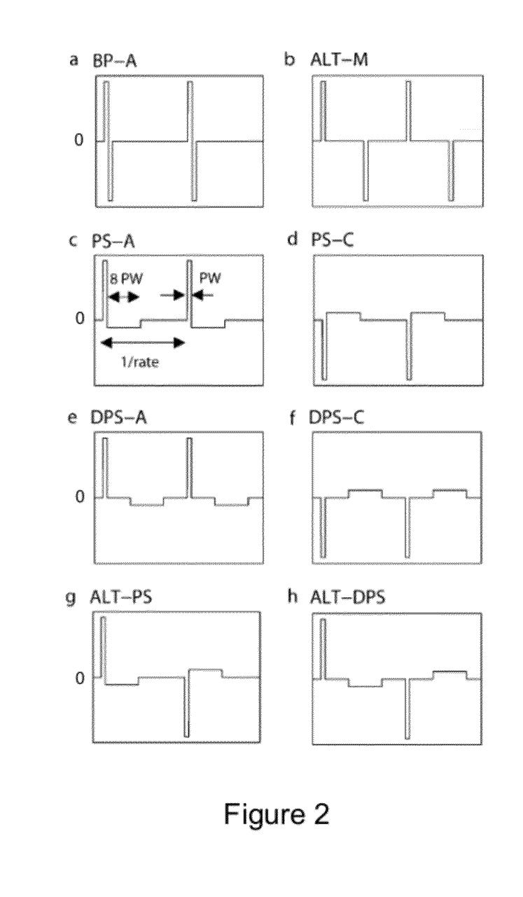

### DETAILED DESCRIPTION OF PREFERRED EMBODIMENTS

We have shown that, at a comfortable listening level, the phase that produces the excitation in monopolar mode is the positive (anodic) phase of the pulse. We now describe how, by manipulating the pulse wave form, in particular using different sorts of asymmetric stimuli, and presenting these in bipolar mode, it is possible: to focus the current at more apical regions of the cochlea than can be achieved with symmetric biphasic pulses in monopolar or bipolar mode, which results in producing a lower place pitch percept to the implant user; to increase the upper limit of temporal pitch perception; to implement a speech processing strategy to use these findings to improve pitch perception in cochlear implant users; and to implement neural steering to produce intermediate place pitch percepts between physical electrodes, for example in devices where only one current source is available.

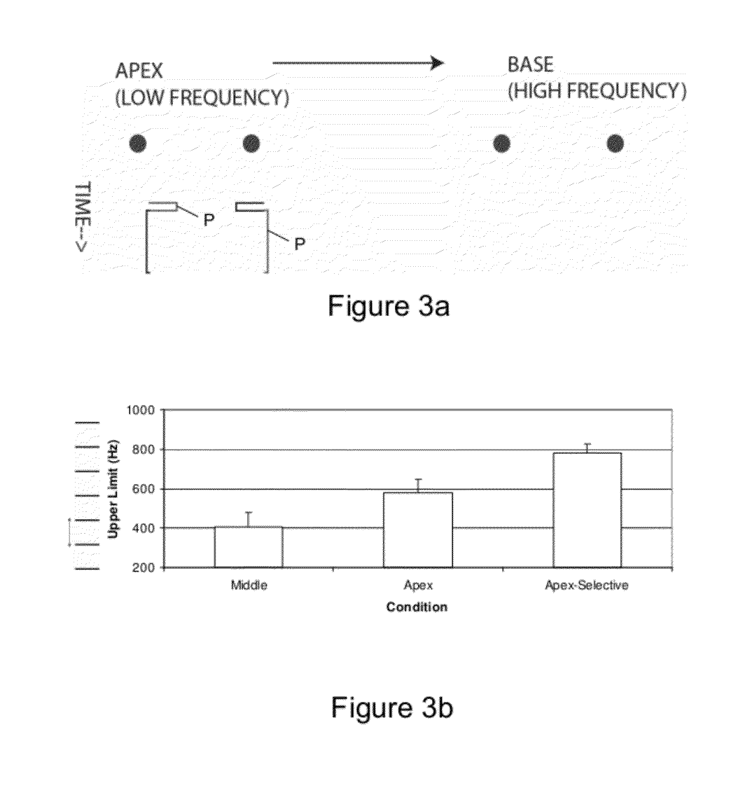

Referring to FIG. 3A, this shows an example in which current is injected via one electrode and returned by its neighbour, so the direction of current flow is always opposite at the two electrodes. In FIG. 3A positive current flow (which we have shown to stimulate auditory neurons) is indicated by “P”. By choosing electrodes at the apex of the cochlea we can stimulate apical neurons, but neurons close to both electrodes will be stimulated. However, a short, high-amplitude pulse will stimulate the nerve more effectively than a long, low-amplitude one, and we can exploit this to selectively excite neurons close to the apical electrode.

FIG. 3B illustrates the results of measuring the upper limit of pitch by stimulating six cochlear implant patients in three ways, at the middle of the cochlea, at the apex of the cochlea, and at the apex of the cochlea using the aforementioned technique which allows more selective stimulation of apical electrodes. It can be seen that an improved upper limit of temporal pitch perception can be achieved by selective stimulation of neurons close to the apical electrode.

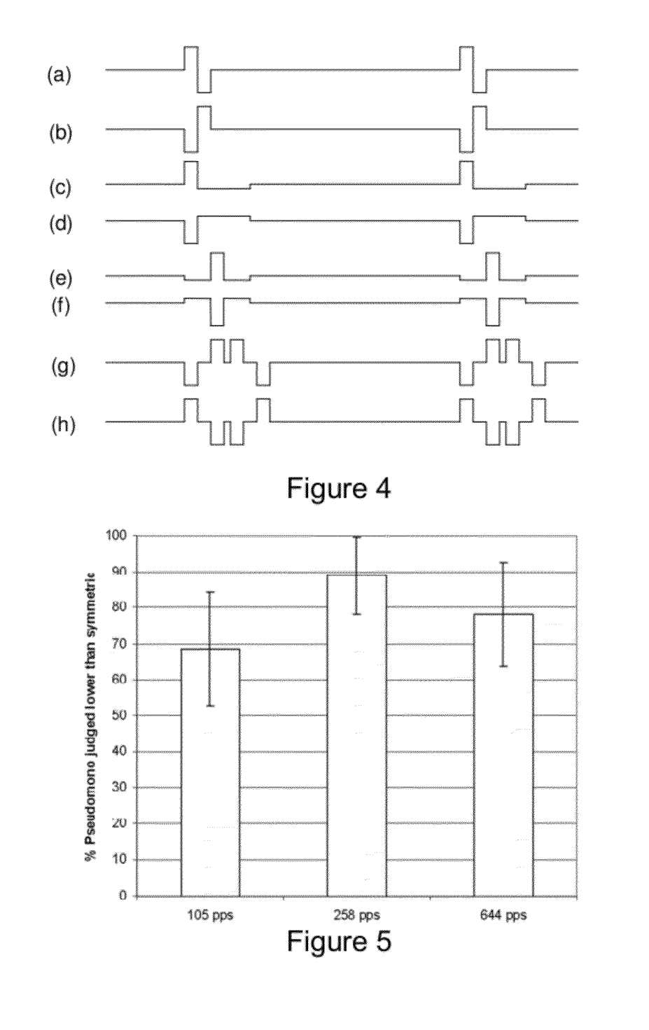

FIGS. 4A and 4B show symmetric biphasic pulses, which have previously been used to provide a charge-balanced electrical stimulation. FIG. 4C shows an anodic pseudomonophasic pulse, and FIG. 4D a cathodic pseudomonophasic pulse (as labelled by the short, high-amplitude phase). An anodic pseudomonophasic pulse is shown in FIG. 3A used to selectively excite neurons close to the apical electrode, but we have obtained the same polarity sensitivity using triphasic pulses with two different cochlear implant devices (Advanced Bionics and Med-El). FIG. 4E shows an anodic triphasic pulse, FIG. 4F a cathodic triphasic pulse; we needed lower current levels to reach MCL (most comfortable level) for anodic triphasic pulses than for cathodic triphasic pulses.

Devices from Cochlear Limited device currently do not allow any other pulse shape than symmetric biphasic. However a Cochlear Limited device allows one to manipulate the polarity order of these pulses. By doing so, we created quadriphasic pulses (FIG. 4g, h) separated by a short inter-pulse gap and found that, when the two middle phases of the pulse were anodic (FIG. 4g), less current (approx. 1.5 dB) was needed to reach MCL than when those phases were cathodic (FIG. 4h).

Bipolar stimulation can be viewed as stimulating simultaneously two adjacent electrodes with opposite-polarity pulses at the same current level. Therefore, by using an asymmetric pulse in bipolar configuration, the pulse will be anodic relative to one electrode and cathodic relative to the other electrode. Because the same current level is being applied to both electrodes, we expect the neurons proximal to the electrode for which the pulse is anodic to be more effectively stimulated than the neurons proximal to the other electrode. Note this does not hold when using standard symmetric biphasic pulses because the first phase is anodic relative to one electrode but the second phase is also anodic relative to the other electrode, we expect both groups of neurons to be affected in the same way.

By presenting the anodic pulse of asymmetric pulses on the most apical electrode of the implant and using a nearby return electrode (here we use an inter-electrode distance of 2.2 mm.), CI (cochlear implant) subjects perceive a lower pitch than when a symmetric biphasic presented on the same two electrodes is used (still in bipolar mode). This is illustrated in FIG. 5 which shows the average scores of five subjects at three different stimulation rates (105, 258 and 644 pps). At the highest two rates, the pseudomonophasic stimulus always was lower in pitch than a symmetric biphasic stimulus. This was less clear at the lowest rate and may be due to the temporal pitch being dominating pitch perception. Furthermore, it produces a lower pitch percept than a monopolar stimulus on the most apical electrode, suggesting that such a technique may extend the lower limit of place pitches available to CI users.

In a different experiment we used pseudomonophasic pulses in bipolar configuration which were anodic relative to the most apical electrode of the electrode array (ie. the most apical electrode received an anodic drive). We asked five Advanced Bionics users to pitch-rank several of these stimuli (differing in their pulse rate, ranging from 105 to 858 pps). The upper limit of temporal pitch was greater than 644 pps for all subjects (geometric mean of 765 pps). We also tested the same subjects with symmetric biphasic pulses both in the middle of the array and at the apex and found an upper limit of 370 pps in the middle (consistent with previous studies of temporal pitch perception in CIs) and of 601 pps at the apex. The only significant differences were that pseudomonophasic apex gave a higher upper limit than both biphasic apex and biphasic middle.

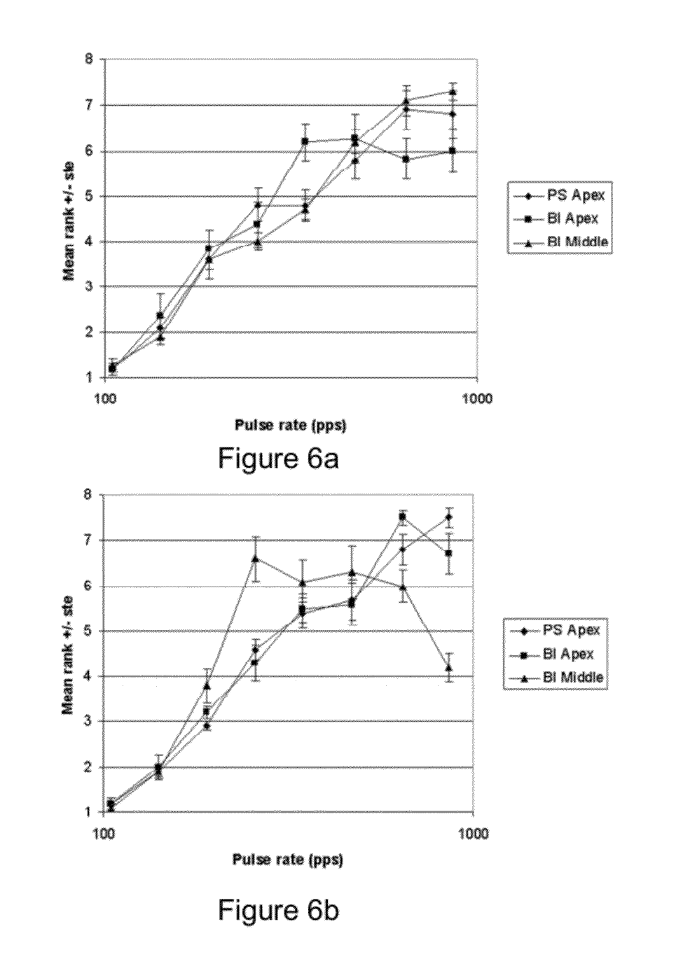

An example of the data obtained with two subjects is shown in FIGS. 6a and 6b. We used the optimally efficient “mid-point comparison procedure” (Long, C. J., Nimmo-Smith, I., Baguley, D. M., O'Driscoll, M., Ramsden, R., Otto, S. R., Axon, P. R. and Carlyon, R. P. (2005). “Optimizing the clinical fit of auditory brain stem implants,” Ear and Hearing 26, 251-262) where subjects had to compare the pitches of 8 different stimuli differing in their pulse rate, on a single channel of the implant. The three conditions (pseudomonophasic apex “PS Apex”, symmetric biphasic apex “BI Apex” and symmetric biphasic middle of the array “BI Middle”) were performed in different blocks. When comparing the upper limit of pitch for symmetric pulses at the apex and in the middle, the two subjects show opposite patterns: S1 (FIG. 6a) is better in the middle while S2 (FIG. 6b) is better at the Apex. However, for pseudomonophasic stimuli (in blue), the two subjects both show high levels of performance.

To exploit the two findings that asymmetric pulses (presented in bipolar mode at the apex) extend the lower range of place pitch experienced by CI users and extend the upper limit of temporal pitch perception, such pulses may be implemented in a speech-processing strategy to extend the relevant limits. Given the present limitations of cochlear implant current sources, such pulses could require long phase durations (in our experiments we used 100 us/phase in BP+1). With current limitations, if such pulses were used to stimulate all channels of the implant, either the stimulation rate would be very low, thereby being deleterious for the delivery of temporal fine structure or simultaneous stimulation would be used. To avoid both of these, one strategy which may be employed is to have asymmetric pulses only on the most apical channel while stimulating all other electrodes in usual monopolar mode with much shorter phase durations.

As previously mentioned, the Advanced Bionics device implements a current-steering strategy, simultaneously stimulating two nearby electrodes. Such a technique requires the implant to have more than one current source, but this is not always available. Here, we provide two techniques to steer the neural excitation between two nearby electrodes. The aim is also to obtain intermediate pitch percepts (place pitch percepts intermediate to those produced by stimulating each electrode individually). Moreover we can provide improved spatial selectivity of the stimulated neurons, and only one current source needed. In embodiments of our system the electrodes can be considered to be stimulated in antiphase ie. when current is flowing into one of the intracochlear electrodes it is flowing out of the other. Also, in embodiments of our system the driven intracochlear electrodes are stimulated by a (single) common current driver (current source or current sink).

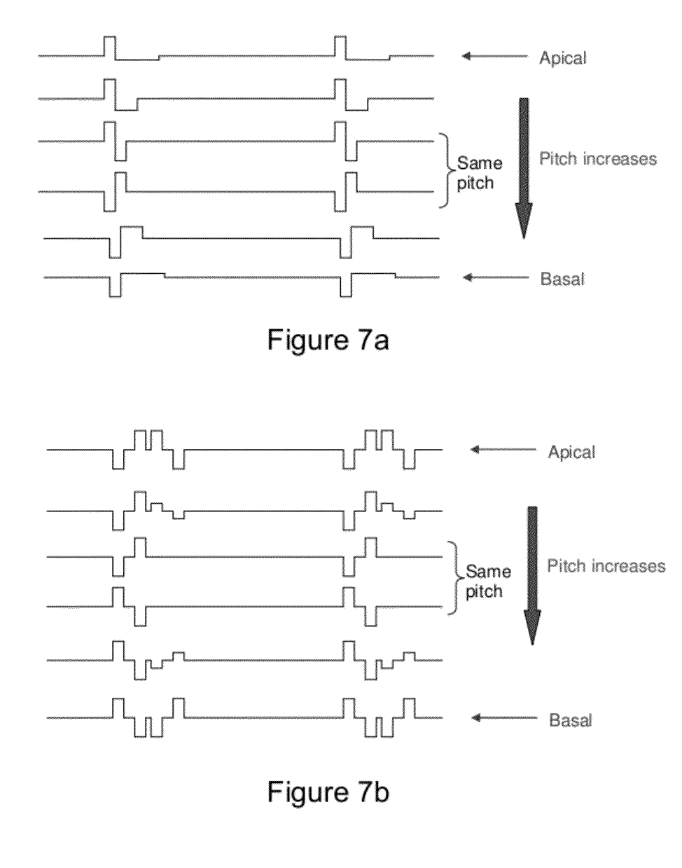

In one approach (FIG. 7a), pseudomonophasic pulses are used. By varying the ratio of duration and amplitude between the two phases, it is possible to elicit different place pitch percepts. It is assumed that there will not be any marked pitch difference between anodic-first and cathodic-first symmetric biphasic pulses. In another, related approach quadriphasic pulses are used (FIG. 7b). By varying the ratio of amplitude of the two consecutive pulses, a similar “neural steering” effect can be obtained.

Referring to FIG. 7A, this shows a progressive variation between a pseudomonophasic pulse and an inverted version of the pulse to change the place pitch percept. At the top of FIG. 7A the pseudomonophasic waveform has a first, short higher amplitude portion and a second, longer, lower amplitude portion of opposite polarity. At the top of FIG. 7A the first portion is positive (anodic) and is applied to a first intracochlear electrode with a second intracochlear electrode acting as the return electrode, the first electrode being closer to an apical position along the electrode array than the second. To increase the place pitch percept the neuron stimulation is moved towards the base of the cochlea as follows: the second portion of the waveform is shortened in time and increased in amplitude (for charge balancing) until a symmetric biphasic pulse waveshape is achieved; this is then inverted (which does not affect the place pitch percept) and the second portion of the waveform (now positive) is progressively extended and reduced in amplitude until the inverted pseudomonophasic waveform at the bottom of FIG. 7A is reached. The waveform at the bottom of FIG. 7A provides cathodic stimulation to the electrode nearest the apex of the cochlea and hence the neural stimulation is moved in a basal direction, thereby increasing the place pitch percept. It can be seen that a progressive change between a pseudomonophasic waveshape (which here includes a delayed pseudomonophasic wave shape) and an inverted pseudomonophasic waveshape can be achieved, if desired. Alternatively discrete points within this progressive change may be selected, for example normal and inverted pseudomonophasic waveshapes, and a symmetric biphasic waveshape.

Referring to FIG. 7B, this also shows progressive change from one version of a waveshape to an inverted version of the waveshape, here using a pair of symmetric biphasic pulses to provide an asymmetric waveshape with greater anodic than cathodic stimulation. Thus referring to the waveshape at the top of FIG. 7B, by providing a pair of symmetric biphasic pulses, the second inverted with respect to the first, an extended duration anodic stimulation is provided (although the overall integrated charge is substantially zero the extended anodic stimulation in the middle portion of the wave form allows the integrated charge to be higher in the anodic than in the cathodic phases). In FIG. 7B the waveshapes are applied to a first (active) electrode and a second electrode is used as a return/reference electrode, the first electrode being closer to the apex of the cochlea than the second (or in a more apical position along the electrode array). In the illustrated example the amplitude of one of the symmetric biphasic pulses is progressively reduced (the example shows the amplitude of the second pulse being reduced, but the amplitude of the first pulse could instead be changed), and since this is a symmetric biphasic pulse, charge balancing is ensured. In implementations, in order to help to maintain a constant loudness across the stimuli, one can change the ratio of amplitude between the two pulses and not only the amplitude of the second pulse. When the amplitude of the (second) symmetric biphasic pulse is substantially zero the other (first) symmetric biphasic pulse may be inverted without substantially changing the place pitch percept. The zero amplitude symmetric biphasic pulse is also inverted (although because its amplitude is zero at this point, this cannot be seen), and the amplitude of this (second) now-inverted pulse is gradually increased until the waveshape shown at the bottom of FIG. 7B is achieved, where once again both symmetric biphasic pulses have substantially the same amplitude. It can be seen in the waveshape at the bottom of FIG. 7B the extended stimulation is cathodic rather than anodic and thus the first electrode, which is the electrode closest to the apex of the cochlea, receives cathodic stimulation and the second, returned/reference electrode effectively receives anodic stimulation, thus moving the location of neural stimulation towards the base of the cochlea thereby increasing the perceived place pitch percept.

No doubt the skilled person will be able to construct variations based on the above example, to move anodic neural stimulation between a first electrode and a second return/reference electrode to thereby steer the place pitch percept by, in effect, steering the location of the maximum difference between anodic and cathodic (integrated) stimulation from the phases of the applied waveshape of the current drive across the intracochlear electrodes.

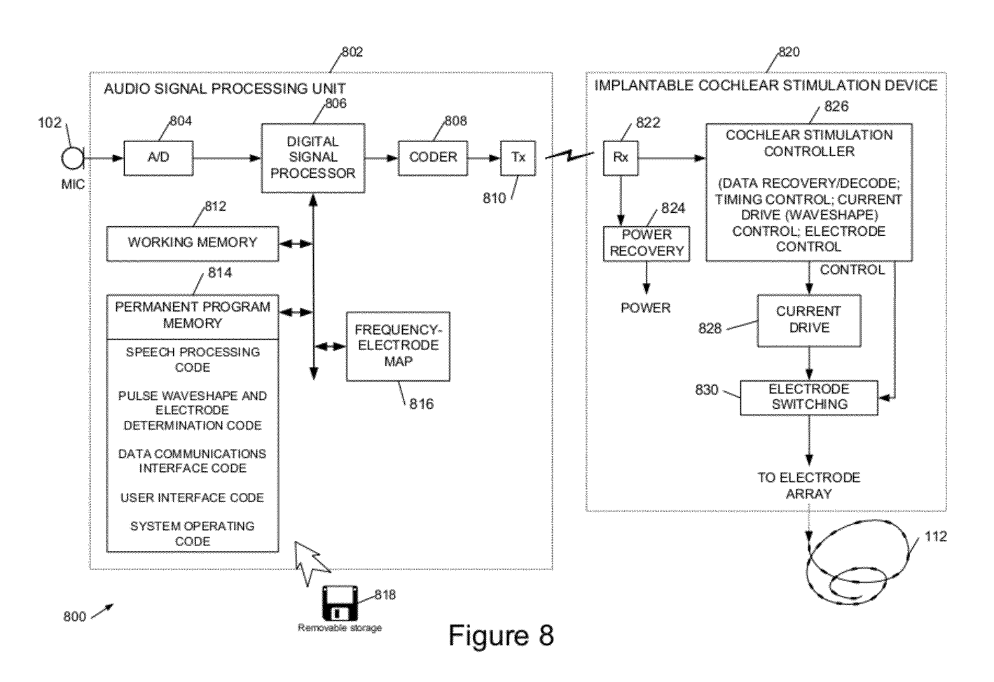

Referring now to FIG. 8, this shows cochlear implant apparatus 800 configured to implement the above described techniques. An audio signal processing unit 802 comprises an analogue-digital converter 804 providing an input to a digital signal processor 806 which has an output to a coder 808 to encode a signal for transmission by transmitter 810. The DSP 806 is coupled to working memory 812 and permanent programme memory 814 storing programme code to implement the above described techniques, and to a frequency-electrode map 816. The code in memory 814 may be provided on a carrier illustratively shown by a removable storage medium 818.

An implantable cochlear stimulation device 820 of the system includes a receiver 822 coupled to a power recovery system 824 to power the device when implanted, and to a cochlear stimulation controller 826, for example implemented on a dedicated integrated circuit. Controller 826 performs functions such a data recovery/decode, pulse timing control, current drive (waveshape) control and electrode switching control, providing corresponding control signals to an adjustable constant current driver 828 comprising one or more current sources (or sinks), which provides an output to an electrode switching arrangement or multiplexer 830, again controlled by controller 826, to drive electrodes with currents of the appropriate waveshapes as requested by the audio signal processing unit. The one or more current drivers may provide controllable output, for example in the range 1 μA to >1 mA at up to 10-20 K pulses per second. The control data from the audio signal processing unit may comprise data packets each specifying, for example, first and second electrodes to which a drive is to be applied, and drive amplitude (and polarity) and duration for each pulse. A back channel may be provided to receive information such as power supply status, electrode impedance and the like. It is preferable to employ a wireless connection between the audio processing and implantable device, but a wired connection may alternatively be employed.

The audio signal processing unit 802 includes speech processing code, for example bandpass filters and compression and/or fundamental frequency identification code and pulse waveshape and electrode determination code (and data) to apply asymmetric bipolar stimulation for extended low frequency place pitch percepts and to drive apical neurons when fundamental frequencies above a threshold are identified in the speech. The code also includes data communications interface code, optional user interface code and background system operating code.

In preferred embodiments the code stored in the non-volatile memory 814 also includes neural steering code to employ neural steering as described above to generate more accurate/finally defined place pitch percepts. For example an increased number of bandpass filters may be employed in the speech processing code and within a pitch range corresponding to an adjacent pair of electrodes neural steering may be employed to place the pitch according to the frequency with the maximum energy within this range or band. In preferred embodiments a frequency-electrode map 816 is also stored in non-volatile memory, for example storing data derived from an initial calibration of the implanted device by an audiologist.

In some embodiments the system may be configured to process speech or music, optionally off-line, to extract the F0 (fundamental frequency) and then input to the implant two “tracks”, for example on a “stereo” connection. In other potentially more preferable embodiments, the implant can simply receive two channels that have been processed by a separate device or computer program.

One such track may comprise the regular speech/music and the other is the F0 track. The F0 track is then used to control the pulses sent to the (temporal) “pitch” channel. This approach may be employed with or separately from the use of asymmetric pulses on the pitch channel. Alternatively the F0 track may be used to control some aspect of the stimulation applied to more than one channel, with other aspects of the stimulation being controlled by the second track. For example a sound signal, which may be music, may be processed in advance, off-line (by a computer program) and the output of this processing may provide two channels of information to the implant, one with F0 and one with “regular” information (for example, data representing the intact sound signal). Thus, for example, a CI (cochlear implant) user may simply buy a CD or download a digital data file (say a .wav or .mp3 file) that has been processed in this way—or alternatively may run an existing sound file through a computer program to produce another sound file—and which they would input to the implant.

We have described the use of pseudomonophasic pulses to excite more apical regions of the cochlea than is possible with biphasic pulses—thereby extending the “place” code to pitch. We have also described how this leads to an improvement in temporal pitch perception. A gap between the two phases of a pseudomonophasic pulse (a “delayed pseudomonophasic” pulse) may be helpful in a speech-processing strategy. This is because a problem with bipolar stimulation can be that you need long phase durations to get up to a comfortable listening level. A gap can alleviate this problem as it makes the stimulation more efficient (by delaying recovery of charge by the second phase). We have shown that advantages of the pseudomonophasic shape can also be achieved using other pulse shapes (FIGS. 4e,f,g,h).

We have further described how to produce pitch percepts that are intermediate between those obtained by stimulating either of two neighbouring electrodes by continuously varying the pulse shape. We call this “neural steering” and is useful, in part because it can be done without needing a separate current source for each electrode—something that can be expensive to implement. In addition, implemented in bipolar mode it can provide a more restrictive current spread than is the case with monopolar mode. We have also described a speech-processing strategy that uses our techniques to improve pitch perception by CI users, in particular by presenting fine timing information (for example, higher fundamental frequencies in speech) to apical channels, more particularly using the novel asymmetric pulse shapes we have described.

In the main the embodiments we have described use bipolar stimulation (returning current via an intracochlear electrode). However, in embodiments this may be “partial bipolar” stimulation, in which a proportion p of the current is returned by an intracochlear electrode and a portion 1-p by an extracochlear electrode. One advantage of this is that one can get to a comfortable loudness with a lower current (at the expense, potentially, of having a wider current spread).

### Further Supporting Data and Examples

### Neural Steering

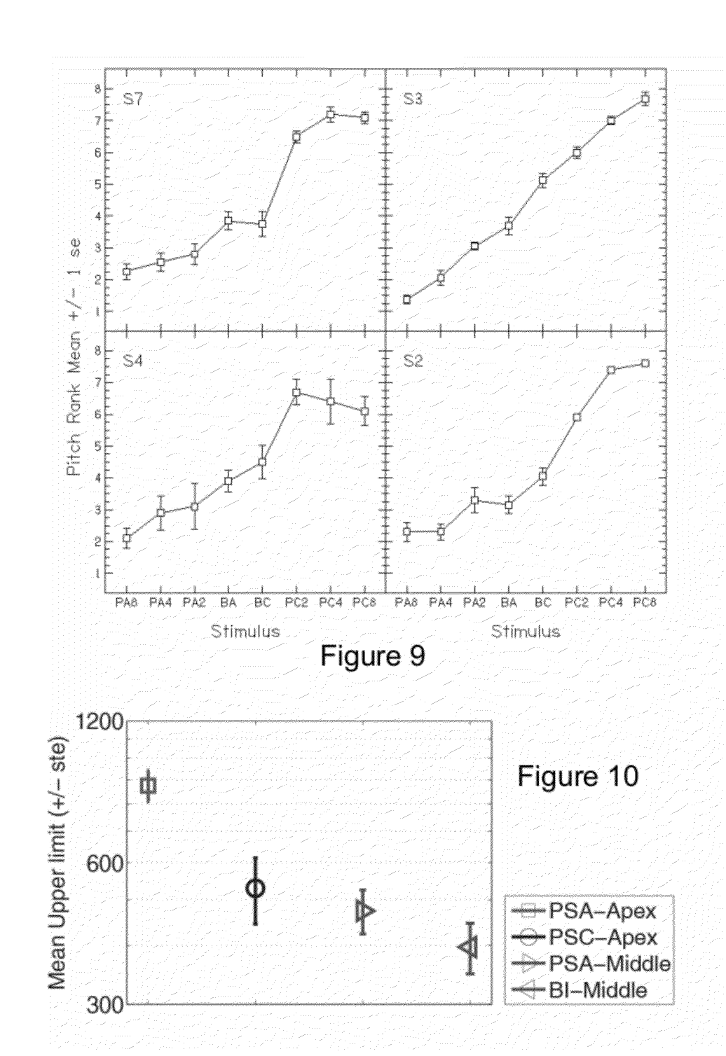

Referring to FIG. 9, this shows pitch ranking results (subjects ranked the pitches of different stimuli) for a range of stimuli similar to those shown in FIG. 7A. In FIG. 9:

PAn=pseudomonophasic pulse with 1^{st} (anodic) phase on more apical electrode, amplitude ratio of two phases=n.
BA=biphasic anodic
BC=biphasic cathodic
PCn=pseudomonophasic pulse with 1^{st} (anodic) phase on more basal electrode, amplitude ratio of two phases=n.

The pitch ranking data were obtained using the Midpoint Comparison Procedure, for stimuli varying continuously in pulse shape. This demonstrates the feasibility of the neural steering approach described above.

### Upper Limit of Temporal Pitch

We have demonstrated that the upper limit of temporal pitch, across conditions, correlates negatively with the current level needed to reach MCL (most comfortable level). The improved upper limit of temporal pitch, obtained with the asymmetric wave shapes described above that produce selective activation at the apex of the cochlea, arises because this form of excitation results in lower MCL values—perhaps due to better neural survival near the apex.

A practical consequence of this finding is that one could identify an electrode or stimulus configuration that produces improved temporal coding simply by measuring the current level needed to obtain MCL in bipolar mode.

### Lower Limit of Place Pitch

FIG. 5, described above, shows that our asymmetric pulse shapes produce a lower place pitch than symmetric pulse shapes, with the mode of stimulation being bipolar in each case. We now have evidence that our asymmetric bipolar stimulation produces a lower place pitch than does symmetric monopolar. The results of a forced-choice experiment conducted with 8 cochlear implant users revealed that the asymmetric bipolar stimulus was judged lower in pitch than the symmetric monopolar stimulus on 82% of trials. This difference was statistically significant.

There is a further potential application for the lower limit of place pitch obtained with our stimuli. Some patients have residual low-frequency hearing, conveyed by neurons innervating the apex of the cochlea. In such patients the electrode array is inserted only part-way into the cochlea; sometimes an especially shortened array is used. The aim is to preserve the residual low-frequency hearing. In practice there is a trade-off between inserting the array far enough to get sufficient electrodes into the cochlea, and not inserting it so far that it damages the residual low-frequency hearing. It may be desirable for the electrical excitation to abut that produced by the residual low-frequency hearing. Embodiments of the methods we describe allow more apical electrical stimulation for a given insertion depth, which helps to address this problem.

### Upper Limit of Temporal Pitch

Referring to FIG. 10, this shows further data on the increased upper limit of temporal pitch. More particularly FIG. 10 shows estimates of the upper limit of temporal pitch for four conditions, all with bipolar stimulation:

PSA-Apex=pseudomonophasic pulses with anodic high-amplitude phase on electrode 1 (most apical) and cathodic high-amplitude phase on electrode 3.
PSC-Apex=as PSA-Apex but with inverted polarity.
PSA-Middle=as PSA-Apex but on two electrodes in the middle of the array
BI Middle=symmetric biphasic waveforms applied to two electrodes in the middle of the array.

FIG. 10 shows estimates of this upper limit obtained from pitch-ranking data from 6 subjects, using the MidPoint Comparison Procedure. The upper limit was obtained by fitting “broken stick” functions to data similar to those shown in FIG. 6. These functions have a rising portion and a flat portion, with the “upper limit” defined as the intercept between these two portions. The greatest upper limit is from PSA-Apex stimulation.

No doubt many other effective alternatives will occur to the skilled person. It will be understood that the invention is not limited to the described embodiments and encompasses modifications apparent to those skilled in the art lying within the spirit and scope of the claims appended hereto.

1-36. (canceled)

37. Cochlear implant apparatus comprising an audio signal processing unit with a wired or wireless coupling to an implantable cochlear stimulation device, wherein said audio signal processing unit has an audio input to receive an audio signal representing a sound, wherein said implantable cochlear stimulation device includes an electrical pulse generator coupled to an electrode array for intracochlear stimulation, wherein the apparatus is configured to apply stimulation to said electrode array to represent said sound, wherein said stimulation comprises application of an electrical pulse across two intracochlear electrodes of said electrode array, and wherein said electrical pulse has a pulse waveshape which is asymmetric under an operation comprising inverting the waveshape about a zero level and then time reversing the waveshape.

38. Cochlear implant apparatus as claimed in claim 37 wherein said audio signal processing unit is configured to control said electrical pulse generator to define said asymmetric waveshape of said electrical pulse.

39. Cochlear implant apparatus as claimed in claim 37 wherein said audio signal processing unit is configured to process said audio signal to determine at least one parameter representing a percept of said sound, wherein said asymmetric pulse waveshape stimulation is used to encode a first set of values of said parameter, and wherein a second, different pulse waveshape is used to encode a second set of values of said parameter.

40. Cochlear implant apparatus as claimed in claim 37, wherein said apparatus is configured to encode a pitch of said sound into one or both of a position of an electrode to which said stimulation is applied and a pulse rate of said bipolar stimulation, and wherein the apparatus is configured to apply one or both of said asymmetric pulse waveshape and bipolar said stimulation selectively to said intracochlear electrodes, such that one of said two stimulated electrodes is a most apical electrode of said electrode array.

41. Cochlear implant apparatus as claimed in claim 37 wherein said audio signal processing unit is configured to process said audio signal to identify high and low frequency perceived place pitch content of said sound, said perceived place pitch being a perceived pitch dependent on a position of stimulation of an electrode along said electrode array, and wherein the apparatus is configured to encode a low frequency said place pitch by selectively applying said asymmetric waveshape to a pair of said intracochlear electrodes including a most apical electrode of said electrode array.

42. Cochlear implant apparatus as claimed in claim 37 wherein said audio signal processing unit is configured to process said audio signal to identify high and low frequency perceived temporal pitch content of said sound, said perceived temporal pitch being a perceived pitch dependent on a rate of stimulation of a said intracochlear electrode, and wherein the apparatus is configured to encode a high frequency said temporal pitch by selectively applying said asymmetric waveshape to a pair of said intracochlear electrodes including a most apical electrode of said electrode array.

43. Cochlear implant apparatus as claimed in claim 37 wherein said audio signal processing unit is configured to process said audio signal to determine a parameter dependent on a perceived place pitch of said sound, and to vary said pulse waveshape of said stimulation between a first version of said waveshape and a second, inverted version of said waveshape dependent on said parameter.

44. Cochlear implant apparatus as claimed in claim 37 wherein said electrical pulse generator is configured to generate said asymmetric waveshape of said electrical pulse.

45. An audio signal processing unit for the cochlear implant apparatus of claim 37, the signal processing unit comprising:
an input to receive an audio signal representing a sound;
an output to provide a control signal to an implanted cochlear stimulation device;
data memory to store audio data for processing;
program memory storing processor control code; and
a digital signal processor, coupled to said program memory to load and implement said code, coupled to said data memory, and coupled between said input and said output to provide said control signal responsive to said audio input to create a perception of said sound in a user; and
wherein said code comprises code for controlling said digital signal processor to generate audio signal representation data defining a representation of said audio signal as a set of pulses for bipolar driving of electrodes of said implanted cochlear stimulation device, wherein said representation data comprises, for a said pulse, a combination of data defining a pulse waveshape and data defining a pair of intracochlear electrodes to which said pulse waveshape is to be applied, wherein said data defining said pulse waveshape comprises data defining a waveshape to said electrode array to represent said sound which is asymmetric under an operation comprising inverting the waveshape about a zero level and then time reversing the waveshape, and to code control said control signal responsive to said audio signal representation data.

46. Cochlear implant apparatus as claimed in claim 37, the apparatus comprising:
a signal input to receive a control signal from an external audio signal processing unit;
at least one pulse generator to generate an electrical pulse;
an electrode array for intracochlear stimulation coupled to said at least one pulse generator; and
a controller coupled to said signal input and to said at least one pulse generator; and
wherein said device is configured to apply stimulation comprising application of said electrical pulse across two intracochlear electrodes of said electrode array, wherein said electrical pulse has a pulse waveshape which is asymmetric under an operation comprising inverting the waveshape about a zero level and then time reversing the waveshape.

47. A method of stimulation of a cochlear implant, the method comprising stimulating a first intracochlear electrode with reference to a second intracochlear electrode using an electrical pulse such that a current flows from said first to said second intracochlear electrode via the cochlea, and wherein said stimulating uses an electrical pulse having waveshape which is asymmetric under an operation comprising inverting the waveshape about a zero level and then time reversing the waveshape.

48. A method of creating a reduced frequency place pitch precept, the method comprising using the method of stimulation of claim 47 to stimulate a most apical electrode of said first and second intracochlear electrodes.

49. A method of creating an increased frequency temporal pitch percept, the method comprising using the method of stimulation of claim 47 to stimulate a most apical electrode of said first and second intracochlear electrodes.

50. A method of stimulation as claimed in claim 47 wherein said first and second intracochlear electrodes comprise electrodes of an electrode array, the method comprising using said asymmetric pulse waveshape to stimulate a most apical electrode of said electrode array and using one or both of: a second, different waveshape, and monopolar electrode stimulation, to stimulate other electrodes of said electrode array.

51. A method of stimulation as claimed in claim 47 used in a method of steering a place pitch percept between two electrodes of a cochlear implant, the method further comprising inverting a polarity of said waveshape of said electrical pulse applied across said first and second intracochlear electrodes dependent on a target percept for said place pitch percept.

52. A method as claimed in claim 51 comprising varying said waveshape progressively between a first polarity and a second, inverted polarity dependent on target said place pitch percept.

53. A method of controlling a cochlear implant using the method of claim 47, the cochlear input having:
an input to receive a control signal from a remote device;
at least one electrical pulse generator, coupled to said input to generate an electrical pulse responsive to said control signal; and
an electrode array, said electrode array comprising a generally longitudinal support bearing electrodes at intervals along said support, to apply said electrical pulse to the cochlea of a user via one or more of said electrodes;
the method comprising:
applying a bipolar said electrical pulse to said cochlea using said electrode array to cause a perception of sound wherein, said applying of said bipolar pulse comprises applying a current drive to a first of said electrodes of said electrode array and providing a return path for said current drive via a second of said electrodes of said electrode array; and
wherein the method further comprises:
using a waveshape for said bipolar electrical pulse which is asymmetric when inverted about a zero drive level and then time reversed.

54. Cochlear implant apparatus as claimed in claim 37 wherein said asymmetric waveshape has a first portion above said zero level and a second portion below said zero level, and wherein said waveshape is configured such that integration of said waveshape results in an excursion of an integrated value of said waveshape away from said zero level which is greater above said zero level than an excursion of said integrated value of said waveshape below said zero level.

55. Cochlear implant apparatus as claimed in claim 37 wherein said asymmetric waveshape comprises a waveshape comprising a first portion above said zero level and a second portion below said zero level, and wherein said first portion is of shorter duration and greater amplitude then said second portion.

56. Cochlear implant apparatus as claimed in claim 55 wherein said first portion of said waveshape is located in time between preceding and following parts of said second portion of said waveshape.

57. Cochlear implant apparatus as claimed in claim 37 wherein said asymmetric waveshape comprises first and second substantially symmetric biphasic waveshapes inverted with respect to one another.

58. Cochlear implant apparatus as claimed in claim 37 wherein said asymmetric waveshape has a first portion above said zero level and a second portion below said zero level and wherein, when applied as bipolar stimulation of said intracochlear electrodes, said stimulation is configured such that said first portion of said waveshape is configured to apply an anodic current to one of said intracochlear electrodes which, when implanted, is closest to an apex of the cochlea.

59. Cochlear implant apparatus as claimed in claim 37 wherein said intracochlear electrodes are separated along said electrode array by 3, 2, 1 or 0 intermediate electrodes or by less than 3.5 mm, 2.5 mm or 1.5 mm.

60. Cochlear implant apparatus as claimed in claim 37 wherein said stimulation comprises bipolar stimulation.

61. A carrier carrying processor control code to, when running, implement the method of claim 47.

62. Cochlear implant apparatus including the carrier of claim 61, wherein said carrier comprises program memory storing said processor control code, the apparatus including an audio signal processing unit, the audio signal processing unit comprising: an input to receive an audio signal representing a sound; an output to provide a control signal to an implanted cochlear stimulation device; data memory to store audio data for processing; and a digital signal processor, coupled to said program memory to load and implement said code, coupled to said data memory, and coupled between said input and said output to provide said control signal in response to said audio input to create a perception of said sound in a user.

63. Cochlear implant apparatus configured to adjust a location of neural stimulation, the apparatus including an audio signal processing unit, the audio signal processing unit comprising: an input to receive an audio signal representing a sound; an output to provide a control signal to an implanted cochlear stimulation device having an intracochlear electrode array; data memory to store audio data for processing; program memory storing processor control code to control operation of the audio signal processing unit; and a digital signal processor, coupled to said program memory to load and implement said code, coupled to said data memory, and coupled between said input and said output to provide said control signal in response to said audio input to create a perception of said sound in a user;
wherein said cochlear implant apparatus is configured to drive first and second intracochlear electrodes of said intracochlear electrode array with a current, and wherein said current drive has an adjustable waveshape to adjust said location of neural stimulation.

64. Cochlear implant apparatus as claimed in claim 63 wherein said current drive is such that a substantially equal and opposite currents flow through said intracochlear electrodes.

65. Cochlear implant apparatus as claimed in claim 63 wherein said current drive waveshape is adjustable between a first version of the waveshape and a second inverted version of the waveshape.

66. Cochlear implant apparatus as claimed in claim 63 wherein said current drive waveshape has first and second portions respectively above and below a zero level of the waveshape, and wherein said current drive waveshape is adjustable to adjust an amplitude and duration of one of said first and second portions with respect to the other subject to a constraint that a total integrated value of the waveshape above said zero level is substantially equal to a total integrated value of the waveshape below said zero level.

67. Cochlear implant apparatus as claimed in claim 63 for adjusting the location of neural stimulation by cochlear implant apparatus, the apparatus comprising means for driving first and second intracochlear electrodes of said intracochlear electrode array with a current drive pulse, and means for adjusting a shape of said current drive pulse to adjust said location of neural stimulation.

68. An audio signal processing unit as claimed in claim 45, the signal processing unit comprising: an input to receive audio signal data representing a sound; an output to provide a control signal to an implantable cochlear stimulation device; data memory to store said audio signal data; program memory storing processor control code; and a digital signal processor, coupled to said program memory to load and implement said code, coupled to said data memory, and coupled between said input and said output to provide said control signal responsive to said audio input to create a perception of said sound in a user; and wherein said code comprises code for controlling said digital signal processor to input or generate audio signal data defining a representation of said sound comprising two channels of data, a first channel representing a fundamental frequency of the input and a second channel representing the original signal or the remainder of the signal; and to send said two channels of data to said cochlear stimulation device to control said device to generate a temporal pitch percept from said first channel of data and to generate a place pitch percept or to convey other information from said second channel of data.

69. The audio signal processing unit of claim 68 in a cochlear implant signal processing system, the cochlear implant signal processing system including a separate processing unit, couplable to said audio signal processing unit, wherein said separate processing unit is configured to generate said two channels of data and to provide said two channels of data to said audio signal processing unit for controlling said cochlear stimulation device

70. A method as claimed in claim 47, the method further comprising generating audio signal data defining a representation of said sound comprising two channels of data, a first channel representing a fundamental frequency of the input and a second channel representing the original signal or the remainder of the signal; and sending said two channels of data to a cochlear stimulation device to control the cochlear stimulation device to generate a temporal pitch percept from said first channel of data and to generate a place pitch percept from said second channel of data.

71. A method as claimed in claim 70 further comprising generating said audio signal data using a separate processing unit couplable to said audio signal processing unit.
